(19) United States
(12) Patent Application Publication
(10) Pub. No.: US 2022/0395402 A1
(43) Pub. Date: Dec. 15, 2022

(54) WEARABLE MEDICAL DEVICES AND RELATED SYSTEMS AND METHODS

(71) Applicant: Orthosensor Inc., Dania Beach, FL (US)

(72) Inventors: Masei Marty TRABISH, Folsom, CA (US); Sanghyun LEE, Seoul (KR); Soohong KIM, Seoul (KR); Miro KANG, Seoul (KR); Seonguk JEON, Seoul (KR)

(73) Assignee: Orthosensor Inc., Dania Beach, FL (US)

(21) Appl. No.: 17/806,050

(22) Filed: Jun. 8, 2022

Related U.S. Application Data
(60) Provisional application No. 63/208,590, filed on Jun. 9, 2021.

(51) Int. Cl. A61F 13/02; A61B 5/00

(52) U.S. Cl. CPC A61F 13/025; A61B 5/6833; A61F 13/0253; A61F 13/0209; A61F 13/0256

(57) ABSTRACT

According to one aspect, a medical device may be configured to couple to a body. The medical device may comprise a material layer; a plurality of adhesive layers coupled to the material layer and configured to couple to a user's skin, wherein each adhesive layer of the plurality of adhesive layers includes a plurality of micro passages; a channel extending between two adjacent adhesive layers of the plurality of adhesive layers; and a superhydrophobic coating covering at least a portion of each of the two adjacent adhesive layers and the material layer forming the channel.

### CROSS-REFERENCE TO RELATED APPLICATIONS

This application claims the benefit of priority under 35 U.S.C. § 119 from U.S. Provisional Application No. 63/208,590, filed on Jun. 9, 2021, which is incorporated by reference herein in its entirety.

### TECHNICAL FIELD

Various aspects of the present disclosure relate generally to medical devices and related methods for coupling a medical device to the surface of the skin, among other aspects. More specifically, at least certain embodiments of the present disclosure relate to devices including an adhesive patch with a plurality of channels and micro holes, among other aspects.

### BACKGROUND

Wearable healthcare devices, specifically those used in fitness and diagnostic medicine, often require small and portable microcontrollers that can take continuous measurements for long periods of time. One frequently used example of such a device is a wearable electrocardiogram (ECG). An ECG is a tool used by physicians to diagnose heart problems and other potential health concerns. Recording sufficient ECG and related physiological data over an extended period of time remains a significant challenge to healthcare providers. An ECG wearable patch may consist of two electrodes (one channel) in a wearable patch, and one microcontroller mounted to the patch. The surface of each electrode may be coated with an non-conductive adhesive and a hydrogel to reduce body contact resistance, and the microcontroller may be surrounded by a protective cover on top of the patch.

Conventionally, maintaining continual contact between ECG electrodes and the skin after a day or two has been a problem. Time, dirt, moisture, and other environmental contaminants, as well as perspiration, skin oil, and dead skin cells from the patient's body, can get between an ECG electrode's non-conductive adhesive and the skin's surface. Long term collection of such material can lead to skin irritation or allergic reactions. Furthermore, all of these factors adversely affect electrode adhesion and the quality of cardiac signal recordings. The physical movements of the patient and their clothing impart various compressional, tensile, and torsional forces on the contact point of an ECG electrode, especially over long recording times, and an inflexibly fastened ECG electrode will be prone to becoming dislodged. Moreover, dislodgment may occur unbeknownst to the patient, making the ECG recordings inaccurate.

Hence, there exists a need for improved wearable patches and other wearable medical devices for patient monitoring. This disclosure seeks to address at least one of the above problems or other problems in the art.

### SUMMARY

Aspects of this disclosure relate to wearable medical devices, and related systems and methods. Each of the aspects disclosed herein may include one or more of the features described in connection with any of the other disclosed aspects.

According to one aspect, a medical device may be configured to couple to a body. The medical device may comprise a material layer; a plurality of adhesive layers coupled to the material layer and configured to couple to a user's skin, wherein each adhesive layer of the plurality of adhesive layers includes a plurality of micro passages; a channel extending between two adjacent adhesive layers of the plurality of adhesive layers; and a superhydrophobic coating covering at least a portion of each of the two adjacent adhesive layers and the material layer forming the channel.

In other aspects, the medical device may include one or more of the following features. The material layer may be a flexible, nonwoven material. Each of the plurality of adhesive layers may be a hydrogel and may include at least one of collagen, gelatin, glycerine, aloe vera, methyl paraben, hydrogenated castor oil, and polyacrylamide/polydopamine (PAM/PDA). The superhydrophobic coating may include one or more of carbon nanofiber, manganese oxide polystyrene (MnO2/PS) nano-composite, zinc oxide polystyrene (ZnO/PS) nano-composite, precipitated calcium carbonate, carbon nano-tube structures, silica nano-coating, fluorinated silanes, and fluoropolymer. Each of the plurality of micro passages may be cylindrical and may have a diameter in the range of 1 μm to 100 μm. Each of the plurality of adhesive layers may be rectangular and spaced from each other adhesive layer of the plurality of adhesive layers. The channel may extend from a first edge of the material layer to a second edge of the material layer, and the first edge may be at an opposite end of the material layer from the second edge. The channel may include a first channel and a second channel, and the first channel may extend transverse to the second channel. The first channel may extend from a first edge of the material layer to a second edge of the material layer, and the first edge may be at an opposite end of the material layer from the second edge; and the second channel may extend from a third edge of the material layer to a fourth edge of the material layer, and the third edge may be at an opposite end of the material layer from the fourth edge. The superhydrophobic coating may cover i) an entire first side surface of a first adhesive layer of the plurality of adhesive layers, ii) an entire second side surface of a second adhesive layer of the plurality of adhesive layers, and iii) a surface of the material layer extending between the first side surface and the second side surface. Each of the plurality of micro passages may extend entirely through at least one of the plurality of adhesive layers. The medical device may further comprise an electronic assembly coupled to the material layer. The electronic assembly may comprise a controller, an antenna, and a power source. At least one electrode may be electronically coupled to the electronic assembly. At least one motion sensor may be electronic coupled to the electronic assembly.

In other aspects, a medical device may be configured to couple to a body. The medical device may comprise a material layer; a plurality of adhesive layers coupled to the material layer and configured to adhere to a user's skin, wherein each adhesive layer of the plurality of adhesive layers is spaced from each other adhesive layer of the plurality of adhesive layers; a plurality of channels, wherein each of the plurality of channels extends between two adjacent adhesive layers of the plurality of adhesive layers; and a superhydrophobic coating covering at least a portion of each of the plurality of channels.

In other aspects, the medical device may include one or more of the following features. Each of the plurality of adhesive layers may include a plurality of micro passages extending entirely through the adhesive layer. Each of the plurality of channels may extend from a first edge of the material layer to a second edge of the material layer. The superhydrophobic coating may include one or more of carbon nanofiber, manganese oxide polystyrene (MnO2/PS) nano-composite, zinc oxide polystyrene (ZnO/PS) nano-composite, precipitated calcium carbonate, carbon nano-tube structures, silica nano-coating, fluorinated silanes, and fluoropolymer.

In other aspects, a medical device may be configured to couple to a body. The medical device may comprise a material layer; an electronic assembly coupled to a first side of the material layer; a plurality of adhesive layers coupled to a second side of the material layer and configured to adhere to a user's skin, wherein each adhesive layer of the plurality of adhesive layers is spaced from each other adhesive layer of the plurality of adhesive layers, and wherein the second side is positioned on an opposite side from the first side; a plurality of channels, wherein each of the plurality of channels extends between two adjacent adhesive layers of the plurality of adhesive layers; and a superhydrophobic coating covering each of the plurality of channels.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 is a perspective view of a wearable medical patch, according to aspects of this disclosure.

FIG. 2 is a perspective view of the bottom portion of the wearable medical patch shown in FIG. 1, according to aspects of this disclosure.

FIGS. 3A and 3B show a side cross-sectional view and a magnified side-cross-sectional view of the medical patch shown in FIG. 1, according to aspects of this disclosure.

FIG. 4 shows a perspective view of an exemplary superhydrophobic coating and the associated contact angle with water and oil, according to aspects of this disclosure.

FIG. 5 shows a perspective view of another embodiment of medical patch, according to aspects of this disclosure.

FIG. 6 shows a perspective view of the medical patch of FIG. 4 with arrows showing the direction of fluid flow through the patch, according to aspects of this disclosure.

FIG. 7 shows an exemplary medical device including the wearable medical patch of FIG. 1, according to aspects of this disclosure.

FIGS. 8A and 8B show another exemplary medical device including the wearable medical patch of FIG. 1, according to aspects of this disclosure.

### DETAILED DESCRIPTION

The present disclosure is drawn to medical patches, systems, devices, and methods for coupling a medical device to a patient, among other aspects. Reference will now be made in detail to aspects of the present disclosure, examples of which are illustrated in the accompanying drawings. Wherever possible, the same or similar reference numbers will be used through the drawings to refer to the same or like parts. The term “coupled to tissue” may refer, for example, to adhering, fixing, attaching, clutching, or fastening, or otherwise secured to a user's body. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.”

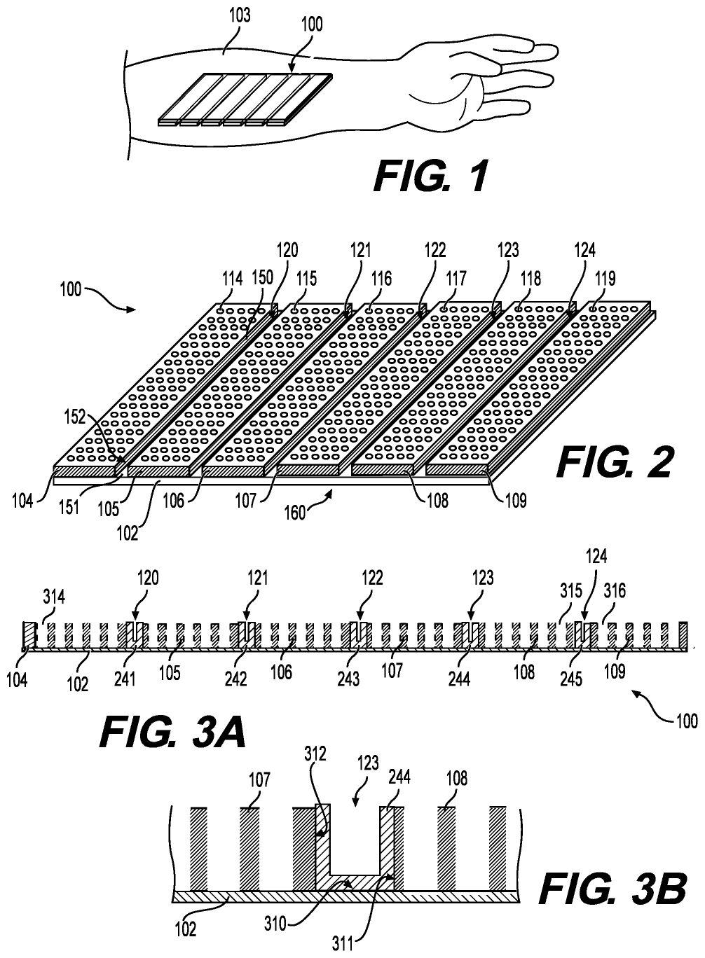

FIG. 1 illustrates a perspective view of a medical patch 100 coupled to a body 103 of a patient. Medical patch 100 may be a wearable patch configured to adhere to tissue via an adhesive. Medical patch 100 may be incorporated into other wearable medical devices, which will be discussed in further detail herein below. As shown in FIG. 1, medical patch 100 may be configured to couple to skin of a patient and may be flexible to facilitate moving with body 103 of the patient. Medical patch 100 may be an adhesive wound covering, such as a bandage, and/or may be used to pull and/or hold portions of tissue together while coupled to a patient's body. Medical patch 100 may be used to protect a portion of skin, such as a rash or other irritated tissue. For example, medical patch 100 may be used to protect a wound, scab, or other tissue from friction, bacteria, physical damage, and/or dirt. Medical patch 100 may be any size, and may be shaped to contour to a patient's body. Medical patch 100 may facilitate maintaining a dry portion of tissue, and may help prevent moisture build-up around an area of tissue proximate to medical patch 100. Medical patch 100 may be incorporated into other medical devices, such as a wearable electrocardiogram (ECG), a wearable hear rate monitor, a wearable motion sensor, a wearable temperature sensor, a wearable calorie tracking device, or other wearable medical device. For example, medical patch 100 may be used as an adhering structure in a wearable ECG, and electronic components may be coupled to medical patch 100.

FIG. 2 illustrates a perspective view of the bottom portion of medical patch 100. Medical patch 100 may include a material layer 102 may be rectangular or any other suitable shape, and one or more adhesive layers 104-109 may be coupled to material layer and configured to couple to skin of a patient. Adhesive layers 104-109 may form rectangular strips extending across material layer 102, and each adhesive layer 104-109 may be spaced from each other adhesive layer 104-109. For example, adhesive layer 105 is shown spaced from adhesive layer 104 and adhesive layer 106. Each of adhesive layers 104-109 may be directly coupled to material layer 102. Channels 120-124 may be formed between the adhesive layers 104-109. For example, channel 120 may be formed between adhesive layer 104 and adhesive layer 105. A side surface 150 of adhesive layer 104, a side surface 152 of adhesive layer 105, and a bottom surface 151 of material 102 may form channel 120. Each of channels 120-124 may extend the entire length material layer 102, and may have a first end at a first edge of material layer 102 and a second end at a second edge of material layer 102. Each of channels 120-124 may be substantially straight and may be configured to receive fluid from the surface of a patient's skin.

Material layer 102 may be a medical woven or non-woven tape, or any other flexible and breathable material. In some examples, material layer 102 may include one or more of cotton, polyester, polypropylene, polyimide, rayon, and polytetrafluoroethylene (PTFE). Material layer 102 may be sterilized, may be treated with anti-microbial agents, may be soft and stretchable, and may either repel or absorb liquids. In some examples; material layer 102 may be a hypoallergenic material. In some examples, material layer 102 may only be permeable in a one direction and may be impermeable in an opposing direction. For example, material layer 102 may be permeable across the surface 151 coupled to each of adhesive layers 104-109 allowing fluid to flow through material layer 102 and through top surface 160, and/or may be impermeable across the top surface 160 not allowing fluid to flow through top surface 160 in a direction towards bottom surface 151.

The one or more adhesive layers 104-109 may include an adhesive hydrogel, and may be configured to adhere to skin of a patient. In other examples, adhesive layers 104-109 may include a plurality of adhesives, such as different types of adhesive hydrogels and/or other forms of adhesive. Adhesive layers 104-109 may include collagen, gelatin, glycerine, aloe vera, methyl paraben, hydrogenated castor oil, polyacrylamide/polydopamine (PAM/PDA), and/or synthetically made materials. Each adhesive layer 104-109 may include a plurality of micro passages 114-119. Each micro passage 114-119 may extend entirely through the adhesive layer 104-109, and may form a grid pattern across the adhesive layer 104-109. Micro passages 114-119 may have a cylindrical shape and may have a diameter in the range of 1 μm to 100 μm (inclusive), or any other suitable diameter. Micropassages 114-119 may be formed via a mold or may be laser cut into adhesive layers 104-109. In some examples, one or more of micro passages 114-119 may contain an air pocket that increases the adhesion of medical patch 100 to skin by acting as a vacuum (via the instant vacuum effect). When medical patch 100 is coupled to a portion of skin of a patient, a first opening of each micro passage 114-119 may be adjacent to and/or abut skin, and adhesive layers 104-109 may couple medical patch 100 to the skin. Micro passages 114-119 may be configured to allow sweat, water, and other liquids to move away from the skin of the patient when medical patch 100 is coupled to the patient's skin. Micro passages 114-119 may increase the flexibility of adhesive layers 104-109 and may facilitate movement of medical patch 100 when coupled to skin of a patient.

Each of channels 120-124 may be coated with a superhydrophobic, or ultrahydrophobic, coating. FIG. 3A shows a side, cross-sectional view of medical patch 100 including material layer 102, adhesive layers 104-109, channels 120-124, and micro passages 314-316. Each of channels 120-124 may be coated with a superhydrophobic coating 241-245. Each superhydrophobic coating 241-245 may cover the surfaces of material layer 102 and adhesive layers 104-109 that form the channel 120-124. FIG. 3B shows a magnified view of medical patch 100 including material layer 102, adhesive layers 107, 108, and channel 123 coated by superhydrophobic coating 244. As shown in FIG. 3B, superhydrophobic coating 244 covers a side surface 312 of adhesive layer 107, a side surface 311 of adhesive layer 108, and a bottom surface 310 of material layer 102. Each of superhydrophobic coatings 241-245 may extend the entire length of each channel 120-124, respectively, and may extend to opposing edges of material layer 102.

Superhydrophobic coatings 241-245 may include carbon nanofiber, manganese oxide polystyrene (MnO2/PS) nano-composite, zinc oxide polystyrene (ZnO/PS) nano-composite, precipitated calcium carbonate, carbon nano-tube structures, silica nano-coating, fluorinated silanes, fluoropolymer, and/or any other superhydrophobic material or combination of materials. Superhydrophobic coatings 241-245 may be applied to channels 120-124 by chemical etching, solution immersion, laser electrodeposition, template deposition, spray coating, or any other application technique known in the art. In some examples, superhydrophobic coatings 241-245 may be applied to an intermediate layer of material (not shown) between i) adhesive layers 104-109 and material layer 102 and ii) the superhydrophobic coating 241-245.

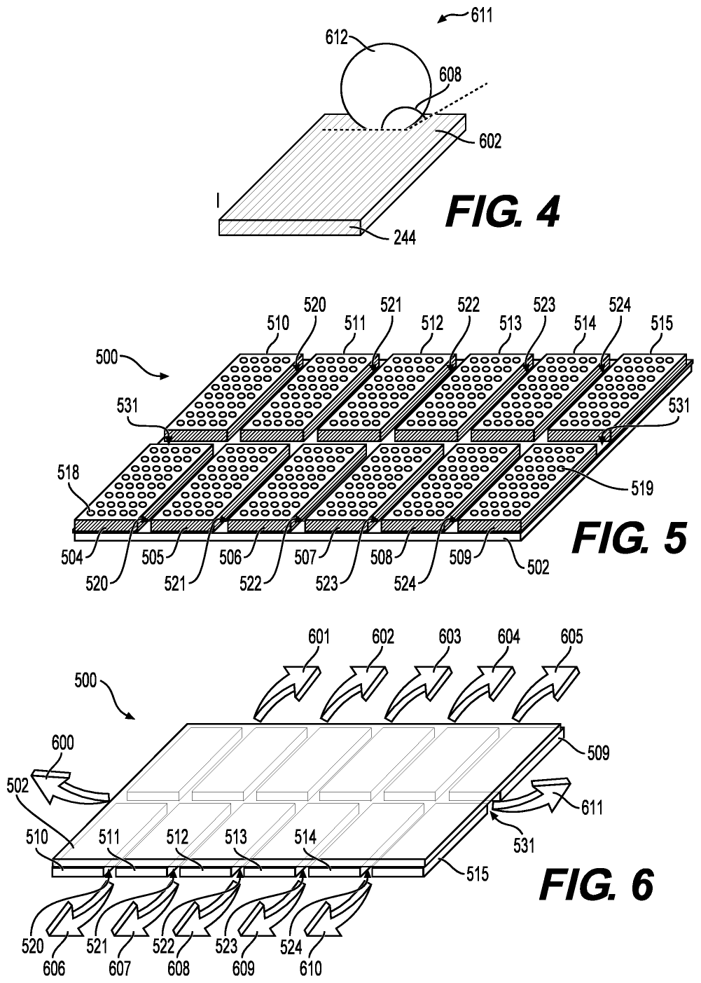

FIG. 4 shows a perspective view of superhydrophobic coating 244 with a water droplet 612 resting on coating 244 and surrounded by gas 611. Coating 244 is shown covering a solid surface 602. The contact angle 608 of coating 244 is the angle formed by a liquid at the three phase boundary where the liquid, gas, and solid intersect. The contact angles of a water droplet on a superhydrophobic material exceed one hundred and fifty degrees. Solid surface 602 has a contact angle 608 of greater than one hundred and fifty degrees and illustrates water droplet 612 interacting with superhydrophobic surface 602. Any of coatings 241-245, and any associated solid surfaces coated by coatings 241-245, may have a contact angle of greater than one hundred and fifty degrees.

FIG. 5 illustrates an alternative embodiment of medical patch 100. Medical patch 500 of FIG. 5 may have many of the same features discussed hereinabove regarding medical patch 100, and common features are indicated with 500 series reference numbers, rather than 100 series reference numbers. Medical patch 500 may include a material layer 502, adhesive layers 504-515 including micro passages 518, 519 in each adhesive layer 504-515, and channels 520-524, and 531. Channel 531 may extend through each of channels 520-524, may be positioned transverse to each of channels 520-524, and may form an array of interconnected channels 520-524, and 531 across medical patch 500. Channel 531 may extend the entire length of material layer 502 from a first edge of material layer 502 to a second edge of material layer 502 opposing the first edge, and each of channels 520-524 may extend from a third edge of material layer 502 to a fourth edge of material layer 502. Each of channels 520-524 may be coated with a superhydrophobic coating in the same manner shown in FIGS. 3A and 3B. FIG. 6 shows fluid flow arrows 600-611 to illustrate the movement of fluid through channels 520-524, and 531 caused by each channel 520-524, and 531 being coated with a superhydrophobic coating. By coating channels 520-524, 531 with a superhydrophobic coating, liquid excreted or collected on the skin of a patient under medical patch 500 may be expelled from underneath medical patch 500 via the superhydrophobic coating in combination with the liquid moving through micro passages 518, 519, which may facilitate reducing moisture collection under medical patch 500 when coupled to a patient's skin.

Although medical devices 100, 500 are shown with straight channels 120-124, 520-524, and 531, other examples may include curved channels or other shapes of channels. In some examples, material layers 102, 502 may be any suitable shape and is not limited to rectangular shapes. The shape of adhesive layers 104-109, 504-515 may be any suitable shape or size, such as circular, oval, polygonal, or irregularly shaped. The arrangement of micro passages 114-119, 518, 519 may vary and is not limited to the rows of passages shown. In some examples, micro passages 114-119, 518, 519 may extend entirely through both adhesive layer 104-109, 504-515 and material layer 102, 502. Each of medical devices 100, 500 may be incorporated into a wearable medical device including electronic components, such as a wearable medical device configured to measure one or more bio signals, such as an ECG.

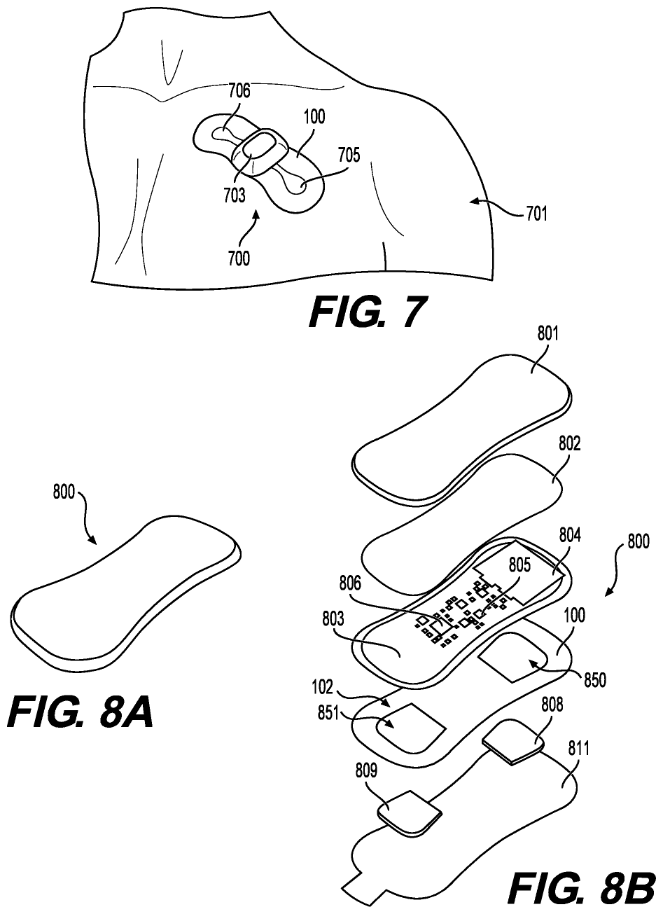

FIG. 7 illustrates an exemplary medical device 700 including medical patch 100. As shown in FIG. 7, medical device 700 is coupled to a body 701 of a patient via medical patch 100. Medical device 700 may include an electronic assembly 703, two electrodes 705, 706, and medical patch 100. Electronic assembly 703 may be coupled to a portion of medical patch 100, and electrodes 705, 706 may be positioned within holes or openings in medical patch 100 to allow electrodes 705, 706 to directly contact the skin of patient 701. Electronic assembly 703 may include a controller, one or more wires, an antenna or other means for wireless electronic communication, and a power source such as a battery. Electronic assembly 703 may be coupled to medical patch 100 via glue or other adhesive. Although medical patch 100 is shown in FIG. 7 with a different form factor than shown in FIGS. 1-3, medical patch 100 shown in FIG. 7 may have any of the features discussed herein in relation to FIGS. 1-3 or any of the features discussed in relation to medical patch 500.

FIG. 8A illustrates an alternative embodiment of medical device 800 including medical patch 100, and FIG. 8B illustrates an exploded view of medical device 800. Medical device 800 may include an elastic polyurethane top layer 801, a foam spacer 802, an electronic assembly 803, medical patch 100, electrodes 808, 809, and a protective liner 811. Medical patch 100 may include holes 850, 851 configured to receive electrodes 808, 809. Electronic assembly 803 may include a controller 806, a flexible antenna 805, and a battery 804. Electronic assembly 803 may be flexible and may be configured to move with a patient's body when coupled to the patient's skin. Each of electrodes 808, 809 may be electrically coupled to electronic assembly 803, and controller 806 may be configured to receive an electrical signal from each electrode 808, 809 and transmit information to a remote system via antenna 805. Protective liner 811 may be configured to be removed prior to application of medical device 800 to skin of a patient. Electronic assembly 803 may be directly coupled to medical patch 100, for example directly coupled to material layer 102.

Medical device 800 illustrates an exemplary embodiment of a wearable medical device incorporating medical patch 100. Medical patch 100 may be used as an adhesive patch in other wearable medical devices known in the art, such as different types of wearable ECG devices. It is to be understood that medical devices 100, 500 can be suitably modified, within the scope of this application, to record a variety of physiological signals. The physiological signal may be at least one of electrocardiogram (ECG), electroencephalogram (EEG), electromyogram (EMG), Electroretinogram (ERG), Electrooculography (EOG), Electroolfactogram (EOG), Electropalatogram (EPG), Electrogastroenterogram (EGEG), Electrocochleography (ECOG), Galvanic skin response (GSR) and any other physiological signal. In some examples, medical devices 100, 500 may be used for wound treatment or protection, such as used in a bandage or band aid to cover a wound. In some examples, medical devices 100, 500 may include one or more motion sensors for monitoring movement of a patient.

The medical devices, systems, and methods discussed herein may provide a long term coupling mechanism for wearable medical devices that may reduce the build up of moisture over time, improve device adhesion to a patient's skin, facilitate drainage of liquid away from the wearable medical device while coupled to a patient, and may facilitate drying the area of a patient's skin that is coupled to the wearable device after exposure to liquid. The medical devices, systems, and methods discussed in this disclosure may help reduce skin irritation or allergic reactions caused by wearable medical devices coupled to a patient's skin. Furthermore, the medical devices, systems, and methods discussed in this disclosure may reduce the likelihood of liquid, collected around a wearable medical device, interfering with electrical components of a wearable medical device, such as short circuiting electrical components.

It will be apparent to those skilled in the art that various modifications and variations can be made to the disclosed system, methods, and devices without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

We claim:

1. A medical device configured to couple to a body, the medical device comprising:
a material layer;
a plurality of adhesive layers coupled to the material layer and configured to couple to a user's skin, wherein each adhesive layer of the plurality of adhesive layers includes a plurality of micro passages;
a channel extending between two adjacent adhesive layers of the plurality of adhesive layers; and
a superhydrophobic coating covering at least a portion of each of the two adjacent adhesive layers and the material layer forming the channel.

2. The device of claim 1, wherein the material layer is a flexible, nonwoven material.

3. The device of claim 1, wherein each of the plurality of adhesive layers is a hydrogel and include at least one of collagen, gelatin, glycerine, aloe vera, methyl paraben, hydrogenated castor oil, and polyacrylamide/polydopamine (PAM/PDA).

4. The device of claim 1, wherein the superhydrophobic coating includes one or more of carbon nanofiber, manganese oxide polystyrene (MnO2/PS) nano-composite, zinc oxide polystyrene (ZnO/PS) nano-composite, precipitated calcium carbonate, carbon nano-tube structures, silica nano-coating, fluorinated silanes, and fluoropolymer.

5. The device of claim 1, wherein each of the plurality of micro passages are cylindrical and have a diameter in the range of 1 μm to 100 μm.

6. The device of claim 1, wherein each of the plurality of adhesive layers are rectangular and spaced from each other adhesive layer of the plurality of adhesive layers.

7. The device of claim 1, wherein the channel extends from a first edge of the material layer to a second edge of the material layer, wherein the first edge is at an opposite end of the material layer from the second edge.

8. The device of claim 1, wherein the channel includes a first channel and a second channel, and wherein the first channel extends transverse to the second channel.

9. The device of claim 8, wherein the first channel extends from a first edge of the material layer to a second edge of the material layer, wherein the first edge is at an opposite end of the material layer from the second edge; and
wherein the second channel extends from a third edge of the material layer to a fourth edge of the material layer, wherein the third edge is at an opposite end of the material layer from the fourth edge.

10. The device of claim 1, wherein the superhydrophobic coating covers i) an entire first side surface of a first adhesive layer of the plurality of adhesive layers, ii) an entire second side surface of a second adhesive layer of the plurality of adhesive layers, and iii) a surface of the material layer extending between the first side surface and the second side surface.

11. The device of claim 1, wherein each of the plurality of micro passages extend entirely through at least one of the plurality of adhesive layers.

12. The device of claim 1, further comprising an electronic assembly coupled to the material layer.

13. The device of claim 12, wherein the electronic assembly comprises a controller, an antenna, and a power source.

14. The device of claim 13, further comprising at least one electrode electronically coupled to the electronic assembly.

15. The device of claim 13, further comprising at least one motion sensor electronic coupled to the electronic assembly.

16. A medical device configured to couple to a body, the medical device comprising:
a material layer;
a plurality of adhesive layers coupled to the material layer and configured to adhere to a user's skin, wherein each adhesive layer of the plurality of adhesive layers is spaced from each other adhesive layer of the plurality of adhesive layers;
a plurality of channels, wherein each of the plurality of channels extends between two adjacent adhesive layers of the plurality of adhesive layers; and
a superhydrophobic coating covering at least a portion of each of the plurality of channels.

17. The device of claim 16, wherein each of the plurality of adhesive layers includes a plurality of micro passages extending entirely through the adhesive layer.

18. The device of claim 16, wherein each of the plurality of channels extends from a first edge of the material layer to a second edge of the material layer.

19. The device of claim 16, wherein the superhydrophobic coating includes one or more of carbon nanofiber, manganese oxide polystyrene (MnO2/PS) nano-composite, zinc oxide polystyrene (ZnO/PS) nano-composite, precipitated calcium carbonate, carbon nano-tube structures, silica nano-coating, fluorinated silanes, and fluoropolymer.

20. A medical device configured to couple to a body, the medical device comprising:
a material layer;
an electronic assembly coupled to a first side of the material layer;
a plurality of adhesive layers coupled to a second side of the material layer and configured to adhere to a user's skin, wherein each adhesive layer of the plurality of adhesive layers is spaced from each other adhesive layer of the plurality of adhesive layers, and wherein the second side is positioned on an opposite side from the first side;
a plurality of channels, wherein each of the plurality of channels extends between two adjacent adhesive layers of the plurality of adhesive layers; and
a superhydrophobic coating covering each of the plurality of channels.
